(19) United States
(12) Patent Application Publication
(10) Pub. No.: US 2006/0165824 A1
(43) Pub. Date: Jul. 27, 2006

(54) Compositions, kits, and methods for enhancing gastrointestinal health

(75) Inventor: Deepa Ashok Khambe, Cincinnati, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 11/043,798

(22) Filed: Jan. 26, 2005

(51) Int. Cl. A61K 36/185

(52) U.S. Cl. 424/769

(57) ABSTRACT

Disclosed herein are compositions, kits, and methods useful for enhancement of gastrointestinal health. For example, compositions comprising matter derived from a plant of the genus Terminalia; and a soluble fiber are disclosed. Also disclosed are kits wherein the extract of the plant matter and the soluble fiber need not be present within a common composition. Methods of enhancing gastrointestinal health are also disclosed.

### FIELD OF THE INVENTION

The present invention relates to compositions, kits, and methods that are useful for enhancement of gastrointestinal health, including treatment of gastrointestinal conditions. The compositions are useful for a variety of treatments including, for example, normalizing bowel function, inducing Taxation, and regularizing gastrointestinal transit times.

### BACKGROUND OF THE INVENTION

Soluble fiber such as psyllium and methylcellulose are known laxatives. Bulk fiber laxatives are often powders which are mixed in water and consumed. These laxatives provide a natural bowel movement with minimal to no diarrhea and a bulked bowel movement. However, bulk laxatives traditionally work relatively slowly and may produce adverse effects such as gas and bloating. An ideal laxative would be one which provides the benefits of bulk fiber, speed to relief and minimal to no adverse effects. The present invention relates to compositions and kits comprising extract of the plant of the genus Terminalia and a soluble fiber for improved laxative and other gastrointestinal health benefits.

### SUMMARY OF THE INVENTION

The present invention is directed to compositions, kits, and methods useful for enhancement of gastrointestinal health. In one embodiment, the invention is directed to a composition comprising:
(a) matter derived from a plant of the genus Terminalia; and
(b) soluble fiber.

In another embodiment, the extract of the plant matter and the soluble fiber need not be present within a common composition. In this embodiment, the invention is directed to kits comprising:
(a) a first composition comprising matter derived from a plant of the genus Terminalia; and
(b) a second composition comprising soluble fiber.

In yet another embodiment, the invention is directed to methods of enhancing gastrointestinal health; these methods may be achieved through administration of the composition comprising the extract and the soluble fiber or, additionally or alternatively, through administration of the first composition and second composition of the kit.

### DETAILED DESCRIPTION OF THE PRESENT INVENTION

Various documents, for example, publications and patents, may be recited throughout this disclosure. All such documents are hereby incorporated by reference.

All percentages and ratios are calculated by weight unless otherwise indicated. All percentages and ratios are calculated based on the total composition unless otherwise indicated.

Referenced herein may be trade names for components including various ingredients utilized in the present invention. The inventors herein do not intend to be limited by materials under a certain trade name. Equivalent materials (e.g., those obtained from a different source under a different name or reference number) to those referenced by trade name may be substituted and utilized in the descriptions herein.

In the description of the invention various embodiments and/or individual features are disclosed. As will be apparent to the ordinarily skilled practitioner, all combination of such embodiments and features are possible and can result in preferred executions of the present invention.

The compositions herein may comprise, consist essentially of, or consist of any of the elements as described herein.

While various embodiments and individual features of the present invention have been illustrated and described, various other changes and modifications can be made without departing from the spirit and scope of the invention. As will also be apparent, all combinations of the embodiments and features taught in the foregoing disclosure are possible and can result in preferred executions of the invention.

### Compositions and Components Utilized in the Present Invention

The present invention is directed to compositions, kits, and methods useful for enhancement of gastrointestinal health. In one embodiment, the composition is directed to a composition comprising:
(a) matter derived from a plant of the genus Terminalia; and
(b) soluble fiber.

The combination of the described extract and the soluble fiber may provide unexpected and synergistic results in terms of improving gastrointestinal health. That is, the combination of the extract with the soluble fiber may enhance efficacy of gastrointestinal movement relative to the extract alone and relative to the fiber alone. This may be consistent even where the extract and soluble fiber are in combination with each other relative to where the extract and soluble fiber are used alone at higher levels. This exciting finding has the potential to revolutionize therapy currently used to enhance gastrointestinal health.

In each of the compositions and kits, matter derived from a plant of the genus Terminalia and a soluble fiber are utilized. These are described for convenience as follows:

Matter Derived from a Plant of the Genus Terminalia

Certain plant species of the genus Terminalia have been used in the Ayurvedic system of medicine. Vernacular names of illustrative plants within the genus Terminalia include the almond, myrobalan, and terminalia. Illustrative members of the genus Terminalia include Terminalia bellerica (sometimes referenced by a common name, myrobalan), Terminalia carolinensis (sometimes referenced by a common name, terminalia), Terminalia catappa (sometimes referenced by a common name, tropical almond), Terminalia chebula (sometimes referenced by a common names, chebulic myrobalan, haritaki, or hirada), Terminalia ivorensis (sometimes referenced by a common name, Ivory Coast almond), Terminalia muelleri (sometimes referenced by a common name, Australian almond), Terminalia myriocarpa (sometimes referenced by a common name, East Indian almond), Terminalia oblonga, and Terminalia superba (sometimes referenced by a common name, superb terminalia).

In accordance with the present invention, matter derived from a plant of the genus Terminalia is utilized. In one embodiment herein, the plant is of the species Terminalia chebula or Terminalia bellerica; in another embodiment, the species is Terminalia chebula. For example, Terminalia chebula and Terminalia bellerica, and Emblica officinalis are key ingredients in a medicinal blend known as Triphala; this composition has a bitter and pungent taste. Use of Triphala is at the heart of Ayurvedic medicine which often promotes health through successive steps of purification and tonification.

The matter derived from the plant should further, in part, be derived from the fruit of the plant. In one embodiment herein, the matter derived from the plant is an extract. For example, the matter may be extracted from at least a portion of the fruit of the plant. Preparation of extracts will be well understood by one of ordinary skill in the art. The extract may be used, for example as a powder or other solid form, or an aqueous or organic extract (such as, alcoholic extract) may be used.

In another embodiment herein, the matter derived from the plant is a powder or other solid. For example, the matter may be derived from any portion of the fruit of plant matter of the genus Terminalia and dried, ground, or otherwise manipulated for ease of use. For example the matter may be provided by obtaining the fruit of the plant, removal of the pit or seed, and grinding the relatively dry fruit pulp to a powder or other solid.

The matter derived from the plant advantageously comprises tannins, whereby at least one tannin is susceptible to hydrolysis to ellagic acid in the presence of gastrointestinal pH conditions.

As an example, the extract may be optionally provided as a powdered composition that is commercially available. For example, extracts of Terminalia chebula may be purchased under the label HARITAKI, by Bazaar of India. Other extracts of the genus Terminalia may be obtained through Ayurceutics Company, Zandu Pharmaceuticals, or Himalaya Drugs, all of India. Alternatively or additionally, the extract may be a lyophilized powder or other solid prepared in accordance with known techniques. Alternatively or additionally, the ordinarily skilled artisan will know to how to prepare aqueous or organic extracts of, for example, dried fruits of plant matter of the genus Terminalia.

The Soluble Fiber

Soluble fiber is well-known to those of ordinary skill in the art. Non-limiting examples of soluble fiber include, but are not limited to, glucomannan (e.g., konjac), oat fiber, pectin, psyllium, psyllium hemicellulose, guar gum, xanthan gum, alginate, gum arabic, fructooligosaccharide (e.g., refined fructooligosaccharide as well as chicory root and inulin), agar, methylcellulose, carrageenan, and mixtures thereof.

In one embodiment herein, the soluble fiber comprises psyllium. Psyllium may be commercially available from The Procter & Gamble Co., Cincinnati, U.S.A. Psyllium hemicellulose will include fractionated fiber as described in U.S. Pat. No. 6,287,609. Fraction B and/or C as described in this patent may be of particular use.

Alternatively or additionally, the soluble fiber comprises psyllium. As an example, fructooligosaccharides are naturally occurring compounds which can be found in a variety of fruits or vegetables including banana, barley, garlic, honey, onion, rye, brown sugar, tomato, asparagus, artichoke, wheat, yacon, or chicory. Fructooligosaccharide may for example be provided as chicory root, as a long chain oligofructose (e.g., inulin), or as refined fructooligosaccharide such as short chain oligofructose (including those in powder or liquid forms). Particularly useful herein are fructooligosaccharide comprising at least one of 1-kestose (abbreviated as GF₂), nystose (GF₃), and IF-beta-fructofuranosylnystose (GF₄). While fructooligosaccharides can be extracted from plants such as those mentioned herein, they can also be formed artificially by adding one, two, or three fructose units to a sucrose molecule by a B-(2-1)-glycosidic linkage of the fructose unit(s) to the fructose unit of sucrose. As an example, fructooligosaccharides are commercially available under the tradename NUTRAFLORA from Golden Technologies Company, Incorporated (which is a short chain oligofructose comprising 1-kestose, nystose, and IF-beta-fructofuranosylnystose. As another example, a mixture of short chain fructooligosaccharide and inulin can be PREBIO1 or a mixture of commercially available RAFTILOSE and RAFTILINE.

Pectin includes those obtained by, for example, hot acidic extraction from citrus peels. Pectin may be obtained, for example, from Danisco Co., Braband, Denmark.

Methylcellullose may also be utilized herein, which is the active component of CITRUCEL, commercially available from GlaxoSmithKline, U.S.A.

Optional Levels of Extract and Soluble Fiber

In particularly preferred embodiments herein, the inventors have found that the compositions herein may comprise the extract and the soluble fiber at a ratio of at least about 1:50, alternatively at least about 1:25, alternatively at least about 1:15, alternatively at least about 1:10, alternatively at least about 1:5, or alternatively at least 1:1, all by weight.

Alternatively or additionally, the compositions herein may comprise at least about 1 milligram, at least about 10 milligrams, at least about 50 milligrams or at least about 100 milligrams of the extract. Alternatively or additionally, the compositions may comprise at least about 100 milligrams, at least 500 milligrams, at least 1 gram or at least 2 grams of the soluble fiber.

### Kits of the Present Invention

In another embodiment, the extract of the plant matter and the soluble fiber need not be present within a common composition. In this embodiment, the invention is directed to kits comprising:
(a) a first composition comprising matter derived from a plant of the genus Terminalia; and
(b) a second composition comprising soluble fiber.

In the embodiments described, at least two separate, distinct compositions are provided. The inventors have discovered that such kits are amenable to compliance with treatment regiments which address issues such as disparate dosing frequencies or timing in accordance with optimized embodiments herein, as well as other like factors. That is, the kits provide opportunity to separately or concurrently dose the first composition comprising the extract and the second composition comprising the soluble fiber, depending for example upon the needs of the mammal for which the kit is intended.

The kits herein comprise the first composition comprising extract and the second composition comprising soluble fiber. Illustrative examples of extract, soluble fiber, and relative levels thereof are as provided herein above.

Alternatively or additionally, for example, the first composition may be provided as a commercially available composition containing the extract, for example, HARITAKI, by Bazaar of India or compositions commercially obtained through Ayurceutics Company, Zandu Pharmaceuticals, or Himalaya Drugs, all of India. Also alternatively or additionally, the second composition may be provided as METAMUCIL, The Procter & Gamble Company, Cincinnati, U.S.A. or may otherwise be provided as FIBERALL or PERDIEM. As another example, wherein the second composition comprises methylcellulose, the methylcellulose may be attained as CITRUCEL, GlaxoSmithKline, U.S.A.

Other first and second compositions may be formulated in accordance with methods which will be well-known to those of ordinary skill in the art.

In accordance with this embodiment, the first and second compositions may be present in the kits as separate compositions, e.g. as separate unit dosage forms which are co-packaged, for example, within a containment device, such as for example a carton, bottle, or the like.

In particularly preferred embodiments of the kits herein, the kits comprise a plurality of unit doses of the first composition and/or a plurality of unit doses of the second composition. Optionally, the number of unit doses of the first composition is the same as the number of unit doses of the second composition. Alternatively and optionally, wherein the kits comprise a plurality of unit doses of both the first and second compositions, the plurality of unit doses of the first composition is less than the plurality of unit doses of the second composition. In another optional embodiment, the number of unit doses of the second composition is from about 2 to about 18 times the number of unit doses of the first composition. In yet another optional embodiment, the number of unit doses of the second compositions is from about 2 to about 4 times the number of unit doses of the first composition. In yet another optional embodiment, the number of unit doses of the second composition is 3 times the number of unit doses of the first composition.

In yet a further embodiment of the present composition, the kits may further comprise information associated with the first and second compositions that use of the kit may or will provide a benefit of enhanced gastrointestinal health. Alternatively or additionally, the information may be that the kit may or will provide a benefit selected from treatment of a condition selected from compromised gastrointestinal motility, constipation (e.g., inducement of Taxation or prokinetic activity including for example gastric emptying), heartburn, non-ulcer dyspepsia, gastroesophageal reflux disease, esophagitis, and combinations thereof.

Preferably, such information indicates that one of the benefits described herein will or may result when the compositions are used in accordance with instructions for use. For example, such directions or instructions for use may include recommended size and frequency of dose and/or any contraindications.

Information need not utilize the actual words used herein, but rather use of words, pictures, symbols, and the like conveying the same or similar meaning are contemplated within the scope of this invention.

### Optional Components and Dose Forms of the Present Compositions and Kits

The compositions described herein (including the first and second compositions of the kits) may be administered concurrently with other compositions, or ingested separately as part of a dosing regimen during a treatment period.

A non-limiting description of suitable excipients and/or other adjuvants is provided in the “Inactive Ingredient Guide” published by the U.S. Food and Drug Administration (see, for example, http://www.fda.gov/cder/drug/iig).

The compositions described herein may be administered in any convenient form including, for example, a capsule, tablet (including swallowable or chewable forms), suspension, suppository, powders (including such powders which are suitable for admixture with a liquid such as, for example, water or juice), wafers, bars, or the like. Wherein the extract and soluble fiber are administered as separate compositions, the unit dose form of each may be independent of the other. For example, the first composition comprising the extract may be in a unit dose form that is a capsule or caplet, while the second composition comprising the soluble fiber may be in a unit dose form which is a capsule or powder.

For oral liquid dosage forms such as suspensions, syrups, elixirs and solutions, acceptable carriers may include for example water, glycol, oil, alcohol, or the like. For solid oral dosage forms such as powders, pills, compressed tablets, hard capsules (starch or gelatin) containing beads, particles or powder of extract and/or soluble fiber, or soft gelatin capsules, carriers may include starch, sugar, kaolin, lubricant, binder, disintegrating agent, or the like. Oral dosage forms can also be film coated, thereby masking any unpleasant taste associated with the extract and/or allowing selective delivery to a desired portion of the gastrointestinal tract.

### Methods of the Present Invention

The present invention is further directed to methods of enhancing gastrointestinal health of a mammal comprising administration of a safe and effective amount of the composition comprising the extract and the soluble fiber. In a further embodiment, the invention is directed to methods of enhancing gastrointestinal health comprising administration of a safe and effective amount of the compositions according to the kits described herein. Gastrointestinal health is health or function of any or all of the gastrointestinal tract of a mammal, such as the esophagus, stomach, small intestine, and the large intestine.

The enhancement of gastrointestinal health of the mammal may include, for example, a benefit selected from treatment of a condition selected from compromised gastrointestinal motility, constipation (e.g., inducement of Taxation or prokinetic activity including for example gastric emptying), heartburn, non-ulcer dyspepsia, gastroesophageal reflux disease, esophagitis, and combinations thereof. For example, administration of the compositions herein may provide an ideal laxative with a natural bowel movement, overnight relief from constipation and reduced side-effects of gas and bloating. Moreover, in addition to the colonic motility, the compositions may regulate gastric emptying and small intestinal motility.

In accordance with the methods herein, a safe and effective amount of the composition(s) are administered herein. As used herein, the term “safe and effective amount” of a composition or like material is an amount that is effective to invoke the desired result in a mammal and is commensurate with a reasonable benefit/risk ratio when used in the manner of this invention. The specific “safe and effective amount” will, obviously, vary with such factors as the particular condition being treated, the physical condition of the treated mammal, the size and weight of the treated animal, the duration of treatment, the nature of concurrent therapy (if any, including concurrent therapy of the first composition with the second composition for example), the specific dosage form to be used, other components present in a given dosed composition, and the dosage regimen desired for the composition.

As used herein, the term “administer,” “administration,” or the like with regard to a particular composition means to provide the composition to the mammal (including oneself) and/or to direct, instruct, or advise the use of the composition for any purpose (preferably, for a purpose described herein). Wherein the administration of one or more of the present compositions is directed, instructed or advised, such direction may be that which instructs and/or informs the user that use of the composition may and/or will provide one or more of the benefits described herein. Non-limiting examples of such instruction or information are set forth herein as part of the description of the present kits.

Administration which is directed may comprise, for example, oral direction (e.g., through oral instruction from, for example, a physician, health professional, sales professional or organization, and/or radio or television media (i.e., advertisement) or written direction (e.g., through written direction from, for example, a physician or other health professional (e.g., scripts), sales professional or organization (e.g., through, for example, marketing brochures, pamphlets, or other instructive paraphernalia), written media (e.g., internet, electronic mail, or other computer-related media), and/or packaging associated with the composition (e.g., a label present on a package containing the composition). As used herein, “written” includes through words, pictures, symbols, and/or other visible descriptors. Such direction need not utilize the actual words used herein, but rather use of words, pictures, symbols, and the like conveying the same or similar meaning are contemplated within the scope of this invention.

Optionally, administration is oral, parenteral or rectal, typically oral. Such methods of administration comprise systemic administration to a mammal (preferably, a human) successive doses of the compositions described herein. In particular, the present methods comprise administering to a mammal in need of treatment a composition comprising the extract and soluble fiber, or separate compositions comprising the first composition (comprising the extract) and the second composition (comprising the soluble fiber). Wherein these separate compositions are administered, administration of each composition may be concurrent or disparate in time.

The methods of the present invention comprise administration (typically, oral) of the compositions either as separate unit doses (e.g., the first composition and the second composition, as described herein above with respect to the kits or concurrently as a single composition (as also described herein), to a mammal (most preferably a human). Frequency of administration is not limited, however, the compositions described herein are typically administered on an infrequent or as-needed basis, at least weekly, at least daily, or on a more or less frequent basis.

For example, the compositions described herein may be administered once daily or with meals. The compositions of the present invention may be administered in the morning hours (including during or subsequent to breakfast) or after dinner. Alternatively or additionally, the compositions may be dosed in the afternoon. Wherein the components are administered separately (such as in the case wherein a kit is utilized, and administration of the first composition and second composition are at least in part disparate in frequency or time), the compositions may be dosed at various times or frequencies.

For example, optionally wherein the kits are utilized, it may be particularly advantageous to dose the second composition comprising the soluble fiber two or three times daily, with or without meals, while the first composition comprising the extract may optionally be administered only once daily. In general, first compositions comprising the extract are administered at least once monthly, or at least once weekly, or at least once daily, or at least twice daily, or at least three times daily. Also in general, second compositions comprising the soluble fiber are administered at least once monthly, or at least once weekly, or at least once daily, or at least twice daily, or at least three times daily.

In one embodiment, at least one unit dose of the second composition comprising the soluble fiber is administered concurrently with the first composition comprising the extract.

### NON-LIMITING EXAMPLES OF THE PRESENT INVENTION

The following are non-limiting examples of the presently described compositions, kits, and methods. The described compositions are prepared utilizing conventional processes to, in the case or separate, distinct composition may be otherwise commercially available. The examples are provided to illustrate the invention and are not intended to limit the scope thereof in any manner.

### Example 1

A kit comprising a plurality of unit doses of each of a first composition and a second composition are prepared containing the following components at indicated amounts:

First Composition: 500 mg of Terminalia chebula fruit pulp powder and carrier, each unit dose packaged in a discrete sachet.

Second Composition: 1 gram of METAMUCL, commercially available in powder form from The Procter & Gamble Company, Cincinnati, U.S.A., each unit dose packaged in a discrete sachet.

### Example 2

A kit comprising a plurality of unit doses of each of a first composition and a second composition are prepared containing the following components at indicated amounts:

First Composition:

| Component | Weight (in mg) |
|---|---|
| Terminalia chebula fruit pulp | 100 |
| powder | |
| Lactose | 100 |
| Maize starch | 50 |
| Polyvinylpyrrolidone | 5 |
| Talcum | 5 |
| Magnesium Stearate | 2 |

The first composition is formed into a tablet, made by conventional processes by mixing the components and compressing those components into tablet form.

Second Composition: 1 gram of CITRUCEL, commercially available in powder form from GlaxoSmithKline, U.S.A., each unit dose packaged in a discrete sachet.

### Example 3

A composition is prepared containing the following components at the indicated amounts:

| Component | Weight % |
|---|---|
| Psyllium Hemicellulose | 25.26 |
| Terminalia chebula fruit pulp powder | 5.26 |
| Maltodextrin | 37 |
| Ultra-Sperse 2000 | 20.25 |
| Tara Gum | 2.70 |
| Aspartame | 0.49 |
| Acesulfame K | 0.49 |
| Citric Acid | 4.44 |
| Flavorants, Colorants, | Balance |
| Sweetners | |

The psyllium hemicellulose is a fractionated fiber as described in U.S. Pat. No. 6,287,609. In this example, the composition is administered by combining about 9.5 grams of the composition with 240 milliliters of water for oral delivery to a human.

### Example 4

A composition is prepared, containing the following components at the indicated amounts:

| Component | Weight % |
|---|---|
| Psyllium | 15.79 |
| Terminalia chebula extract | 5.26 |
| Maltodextrin | 21.65 |
| Citric Acid | 2 |
| N LITE LP (starch, commercially available from | 34.1 |
| National Starch and Chemical, Bridgewater, NJ) | |
| TEXTRA PLUS (starch, commercially available from | 20.2 |
| National Starch and Chemical, Bridgewater, NJ) | |
| Flavorants, Colorants, sweeteners | Balance |

The composition is administered by combining approximately 9.5 grams of the composition with 240 milliliters of water.

### Example 5

A composition is prepared, containing the following components at the indicated amounts:

| Component | Weight % |
|---|---|
| Psyllium | 15.79 |
| Terminalia belerica extract | 5.26 |
| Maltodextrin | 21.65 |
| Citric Acid | 2 |
| N LITE LP (starch, commercially available from | 34.1 |
| National Starch and Chemical, Bridgewater, NJ) | |
| TEXTRA PLUS (starch, commercially available from | 20.2 |
| National Starch and Chemical, Bridgewater, NJ) | |
| Flavorants, Colorants, sweetners | Balance |

The composition is administered by combining approximately 9.5 grams of the composition with 240 milliliters of water.

What is claimed is:

1. A composition comprising:
(a) matter derived from a plant of the genus Terminalia; and
(b) soluble fiber.

2. The composition according to claim 1 wherein at least a portion of the soluble fiber is selected from the group consisting of glucomannan, oat fiber, pectin, psyllium, psyllium hemicellulose, guar gum, xanthan gum, alginate, gum arabic, fructooligosaccharide, agar, methylcellulose, carrageenan, and mixtures thereof.

3. The composition according to claim 2 wherein the extract comprises a material derived from at least a portion of the fruit of the plant matter.

4. The composition according to claim 2 wherein the ratio of the extract to the soluble fiber is at least about 1:50, by weight.

5. The composition according to claim 4 wherein the ratio of the extract to the soluble fiber is at least about 1:25, by weight.

6. The composition according to claim 4 comprising at least about 50 milligrams of the extract and at least about 1 gram of the soluble fiber.

7. The composition according to claim 6 comprising at least about 100 mg of the extract and at least about 2 grams of the soluble fiber.

8. The composition according to claim 4 comprising matter derived from a plant of Terminalia chebula.

9. The composition according to claim 8 wherein at least a portion of the soluble fiber is selected from the group consisting of psyllium, psyllium hemicellulose, methylcellulose, and mixtures thereof.

10. The composition according to claim 9 wherein the extract comprises a material derived from at least a portion of the fruit of the plant matter.

11. The composition according to claim 4 comprising matter derived from a plant of Terminalia belerica.

12. A kit comprising:
(a) a first composition comprising matter derived from a plant of the genus Terminalia; and
(b) a second composition comprising soluble fiber.

13. The kit according to claim 12 wherein at least a portion of the soluble fiber is selected from the group consisting of glucomannan, oat fiber, pectin, psyllium, psyllium hemicellulose, guar gum, xanthan gum, alginate, gum arabic, fructooligosaccharide, agar, methylcellulose, carrageenan, and mixtures thereof.

14. The kit according to claim 13 wherein the extract comprises a material derived from at least a portion of the fruit of the plant matter.

15. The kit according to claim 13 wherein the ratio of the extract to the soluble fiber is at least about 1:50, by weight.

16. The kit according to claim 15 wherein the ratio of the extract to the soluble fiber is at least about 1:25, by weight.

17. The kit according to claim 15 comprising at least about 50 milligrams of the extract and at least about 1 gram of the soluble fiber.

18. The kit according to claim 15 comprising at least about 100 mg of the extract and at least about 2 grams of the soluble fiber.

19. The kit according to claim 15 comprising matter derived from a plant of Terminalia chebula.

20. The kit according to claim 19 wherein at least a portion of the soluble fiber is selected from the group consisting of psyllium, psyllium hemicellulose, methylcellulose, and mixtures thereof.

21. A method of enhancing gastrointestinal health of a mammal comprising administration of a safe and effective amount of the composition according to claim 1 to the mammal.

22. The method according to claim 21 wherein the enhancement of gastrointestinal health comprises treatment of a condition selected from the group consisting of compromised gastrointestinal motility, constipation, heartburn, non-ulcer dyspepsia, gastroesophageal reflux disease, esophagitis, and combinations thereof.

23. The method according to claim 22 wherein the administration is oral.

24. The method according to claim 23 wherein the administration is at least once weekly.

25. The method according to claim 24 wherein the administration is at least once daily.

26. The method according to claim 24 wherein the mammal is a human.

27. A method of enhancing gastrointestinal health of a mammal comprising administration of a safe and effective amount of the compositions according to the kit of claim 12 to the mammal.

28. The method according to claim 27 wherein the enhancement of gastrointestinal health comprises treatment of a condition selected from the group consisting of compromised gastrointestinal motility, constipation, heartburn, non-ulcer dyspepsia, gastroesophageal reflux disease, esophagitis, and combinations thereof.

29. The method according to claim 28 wherein the administration is oral.

30. The method according to claim 29 the first composition and the second composition are each administered at least once weekly.

31. The method according to claim 30 wherein the first composition and the second composition are each administered at least once daily.

32. The method according to claim 30 wherein the mammal is a human.
